(19) United States
(12) Patent Application Publication
(10) Pub. No.: US 2007/0225576 A1
(43) Pub. Date: Sep. 27, 2007

(54) Wireless ambulatory gastrointestinal monitoring system

(75) Inventors: William Brown, Fountain Valley, CA (US); Brad Westcott, Fountain Valley, CA (US); Khai Si Luong, Fountain Valley, CA (US); Jeff Sawyer, Fountain Valley, CA (US)

Correspondence Address: CROCKETT & CROCKETT, 24012 CALLE DE LA PLATA, SUITE 400, LAGUNA HILLS, CA, 92653, US

(73) Assignee: Alpine BioMed Corp.

(21) Appl. No.: 11/455,020

(22) Filed: Jun. 15, 2006

Related U.S. Application Data
(60) Provisional application No. 60/691,151, filed on Jun. 15, 2005.

(51) Int. Cl. A61B 5/103; A61B 5/05; A61B 5/00

(52) U.S. Cl. 600/301; 128/903; 600/593; 600/350

(57) ABSTRACT

An wireless ambulatory reflux monitoring system capable of monitoring and recording esophageal biological parameters during an ambulatory period. The wireless ambulatory reflux monitoring system is further capable of recording perceived reflux events at the discretion of a patient contemporaneously with the recording of biological data.

This application claims priority to U.S. Provisional Patent Application 60/691,151, filed Jun. 15, 2005.

### FIELD OF THE INVENTIONS

The inventions described below relate the field of

### BACKGROUND OF THE INVENTIONS

Gastroesophageal reflux disease (GERD) refers to the abnormal reflux of gastric contents into the esophagus. Normally, stomach acid and digestive enzymes are prevented from flowing backwards into the esophagus by a valve called the lower esophageal sphincter (LES). In GERD patients, this valve is impaired and the symptoms of heartburn and regurgitation develop due to chronic exposure of the esophagus to the irritating contents of the stomach. GERD sufferers often endure significant heartburn and acid regurgitation among other symptoms including: hoarseness, chronic cough, asthma, dental erosions, and nocturnal choking. GERD sufferers also have higher risks of developing esophageal cancer.

Some patients with symptomatic GERD are effectively treated with proton pump inhibitors (PPIs) to reduce gastric acid secretion. When drug therapy fails to control patient symptoms or when patients refuse to take medication, anti-reflux surgery is an option. Fundoplication is the standard surgical treatment for GERD, and entails wrapping the stomach around the LES in order to support the weakened valve.

Monitoring of esophageal pH is the most reliable method of diagnosing GERD. pH monitoring measures the basic pathophysiologic problem of GERD, the exposure time of the esophagus to excessive acid reflux. The amount of time that the esophagus contains acid is determined by a test called a 24-hour esophageal pH test. The pH test is most often performed when drug therapy fails or when surgical options are being considered.

For this test, a catheter is passed through the nose so that the distal tip is positioned in the esophagus. On the tip of the catheter is the pH electrode. The pH electrode is placed 5 cm above the superior margin of the LES. The validity of the pH measurement is dependent on the proper positioning of the electrode and is best accomplished by using manometric (pressure measurement) methods. The proximal portion of the catheter exits from the nose, wraps back over the ear, and runs down to the waist, where it is attached to a recorder. Each time acid refluxes back into the esophagus from the stomach, it stimulates the sensor and the recorder records the episode of reflux. After about 24 hours, the catheter is removed and the record of reflux from the recorder is analyzed. Though this method is useful in obtaining data, it is also burdensome and uncomfortable. Existing wireless systems comprise encapsulated measuring devices that are inflexible and uncomfortable when disposed within the patient.

Other testing methods used to evaluate the symptoms of GERD include monitoring non-acidic reflux and esophageal pressure. Non-acidic reflux monitoring is typically performed by taking electrical impedance measurements. Changes in impedance are used to measure differences in intraluminal esophageal contents. This technique allows detection and quantification of non-acidic bolus movement by using multiple impedance measuring sites.

The measurement of esophageal pressure, known as esophageal manometry, is also useful in evaluating the symptoms of GERD. During esophageal manometry an examination of the esophagus is performed through the use of a small flexible catheter with pressure sensors disposed on the distal section of the catheter. The distal section of the catheter is inserted through the nose, down the back of the throat, and into the esophagus. When the muscles of the esophagus contract, a pressure wave called “peristalsis” is generated within the esophagus and is detected by the sensors on the catheter. The proximal section of the catheter protrudes out the nostril of a patient and is attached to a recorder having a monitoring system that records the pressure. A typical test entails measuring the pressure at the lower esophageal sphincter for about 30 minutes, while the patient occasionally swallows sips of water.

Though the use of manometry, pH and impedance are beneficial in evaluating the symptoms of GERD, there is no current method or system available to obtain the benefits of combining the use these methods in a single testing method and device while recording the data in a less burdensome and invasive manner. A method and system are needed that combines the benefits of integrating pressure manometry with that of pH and impedance measurement while extending pressure measurement beyond the conventional 20-30 minute testing period in a less burdensome manner to the patient.

### SUMMARY

The wireless ambulatory reflux monitoring system integrates the monitoring of acidic and non-acidic reflux parameters with the measuring of esophageal pressure for recording and analyzing gastroesophageal reflux. The wireless ambulatory reflux monitoring system comprises an indwelling sensor array for measuring changes in esophageal impedance, measuring esophageal pH levels and measuring esophageal pressure, a wireless transmitter within the indwelling sensor array, and a data acquisition and recording module having a wireless receiver. The wireless ambulatory reflux monitoring system is an indwelling system that utilizes an indwelling sensor array for measuring multiple parameters within the esophagus and communicates wirelessly to the data acquisition and recording module carried by a patient. Improvement in diagnosis and treatment of GERD as well as reduction in total treatment costs can be achieved through use of the system.

### BRIEF DESCRIPTION OF THE DRAWINGS

FIG. 1 illustrates a patient's upper gastrointestinal tract.

FIG. 2 depicts the indwelling sensor array of the wireless ambulatory reflux monitoring system.

FIG. 3 illustrates the data acquisition and recording module.

FIG. 4 illustrates the wireless ambulatory reflux monitoring system in use.

FIG. 5 depicts the indwelling sensor array attached to the esophagus.

### DETAILED DESCRIPTION OF THE INVENTIONS

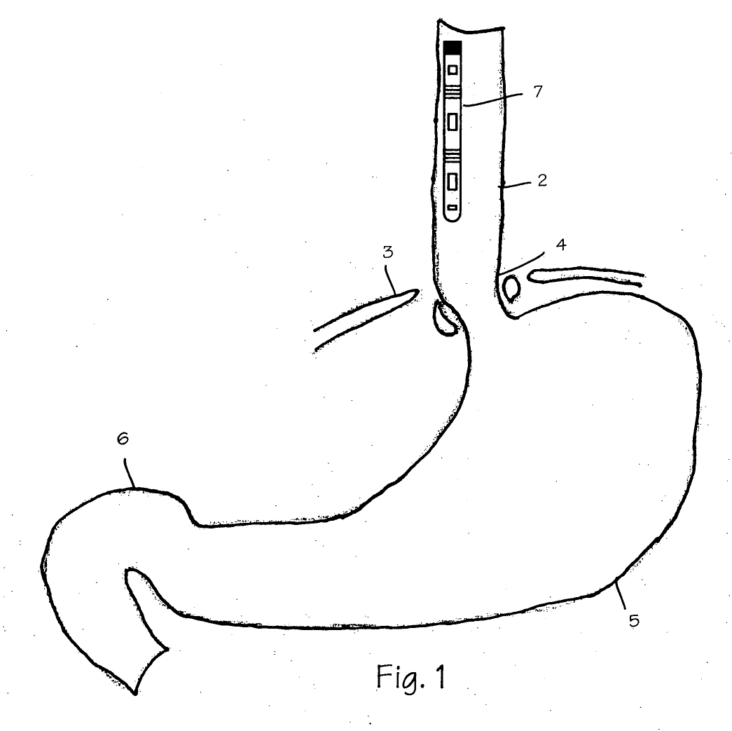

FIG. 1 illustrates a patient's upper gastrointestinal tract 1. The esophagus 2, diaphragm 3, lower esophageal sphincter 4, stomach 5 and duodenum 6 are shown to illustrate use of the wireless ambulatory reflux monitoring system. The wireless ambulatory reflux monitoring system comprises an indwelling sensor array 7 for measuring changes in esophageal impedance, measuring esophageal pH levels and measuring esophageal pressure and a external data acquisition and recording module 8 having a wireless receiver.

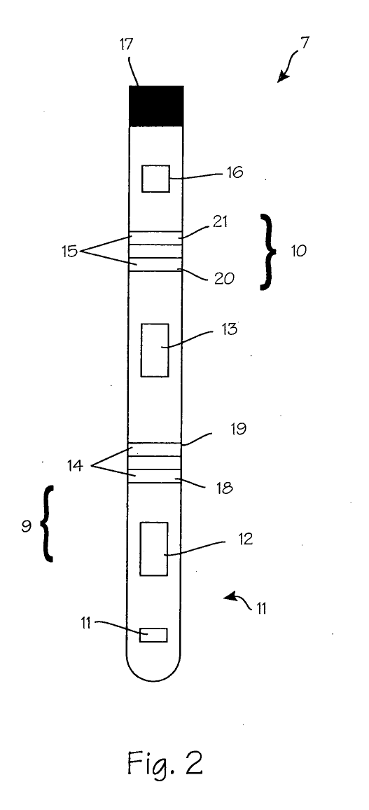

FIG. 2 depicts the indwelling sensor array of the wireless ambulatory reflux monitoring system. The indwelling sensor array is an elongated flexible structure such as a tube or shaft characterized by a distal section 9 and a proximal section 10. The indwelling sensor array may be approximately five to fifteen centimeters in length and approximately five to ten French in diameter. The flexibility of the structure allows the indwelling sensor array to flex and bend with the esophagus of the patient while indwelt. The indwelling sensor array comprises a pH sensor 11, a first pressure sensor 12, a second pressure sensor 13, a first electrode pair 14, a second electrode pair 15, a control system 16 and a wireless transmitter 17. Additional sensors or monitors may include thermometers, gas monitors for detecting the levels of gases such as oxygen and carbon dioxide, or chemical monitors for detecting the presence of different ions.

The control system 16 is capable of receiving biological data from the pressure sensors 12 and 13, pH sensor 11 and the electrode pairs 14 and 15 and sending the biological data using the wireless transmitter 17. A battery, which supplies power to the control system 16, wireless transmitter 17, electrodes and sensors, may be disposed anywhere in the array 7. The control system 16 may employ any suitable microprocessor and any suitable wireless transmitter and wireless protocol.

The distal section 9 of the indwelling sensor array contains the pH sensor 11. The pH sensor 11 comprises an electrode and is in electrical communication with the control system disposed within the indwelling sensor array. The pH sensor is able to generate pH signals in response to relative hydrogen ion concentrations. The pH sensor measures the pH level in the esophagus 2 when the indwelling sensor array 7 is disposed within the esophagus. Measurements from the pH sensor are taken and sent to the control system that sends the data using the wireless transmitter to the data acquisition and recording module.

A first electrode pair 14 is disposed on the distal section 9 of the indwelling sensor array 7 and a second electrode pair is disposed on the proximal section 10 of the indwelling sensor array. The first electrode pair comprises ring 18 and ring 19 of conductive material disposed on the indwelling sensor array. Similarly, the second electrode pair 15 comprises ring 20 and ring 21 of conductive material disposed on the indwelling sensor array. Electrodes 18, 19, 20 and 21 are placed in electrical communication with a control system 16 disposed within the indwelling sensor array. The first electrode pair 14 and second electrode pair 15 are used to measure impedance. The first and second electrode pairs are typically placed approximately one inch from each other.

By analyzing which pair of rings first show a change in impedance, the direction of flow of the measured material in the esophagus can be determined. This allows distinguishing between a swallow of saliva or other material that moves down the esophagus and gastroesophageal reflux, which moves up the esophagus. When gastric juice breaks the barrier of the lower esophageal sphincter and rises in the esophagus, the change in impedance is first registered between rings 18 and 19 and subsequently between the next set of rings 20 and 21 proximal to, and higher up the esophagus than, the first said set of rings 18 and 19. Comparatively, swallowed material moving down the esophagus will first reach the higher set of rings 20 and 21 and will cause a change in impedance between these rings, before a change in impedance between the lower rings 18 and 19 can be seen. It is in this way that it is possible to determine the direction of flow of material in the esophagus. This method distinguishes gastroesophageal reflux, which moves up the esophagus, from swallowed materials, which move down the esophagus, regardless of the pH of materials in the esophagus.

The distal section 9 of the indwelling sensor array further comprises a first pressure sensor 12 and the proximal section 10 of the indwelling sensor array further comprises a second pressure sensor 13. The first pressure sensor is used for measuring esophageal pressure at the distal section of the indwelling sensor array while the second pressure sensor is used for measuring esophageal pressure at the proximal section of the indwelling sensor array. The pressure sensors comprise pressure transducers in electrical communication with a control system disposed within the catheter. The pressure transducer may comprise a solid state or microchannel transducer. The pressure sensors measure the pressure within the esophagus. The pressure measurements are sent to the control system and transmitted to the data acquisition and recording module by the wireless transmitter. The pressure measurements are used to evaluate LES pressure and peristalsis generated within the esophagus. The pressure sensors are also used during placement of the indwelling sensor array.

Traditionally, esophageal manometry would precede pH probe placement and monitoring to identify the location of the LES. This technique has proven to be both time-consuming and uncomfortable for the patient. The pressure sensors of the wireless ambulatory reflux monitoring system allow for accurate placement of the indwelling sensor array. Locating the LES 4 is possible by observing the LES 4 respiration phasic pressures. The location of the LES corresponds to the junction of the esophagus with the diaphragm. The LES 4 is effectively co-planar with the diaphragm. With each inhalation, the diaphragm pushes down into the abdomen creating a partial vacuum in the chest cavity. At the same time, the intra-abdominal pressure rises slightly. With exhalation, the process reverses. By monitoring relative pressure changes instead of mean pressure, the junction of the diaphragm and the esophagus can be determined, thus determining the LES 4 location. This technique is particularly useful for positioning the indwelling sensor array as pressure measurements from within the esophagus can be take by pressure sensors 12 and 13.

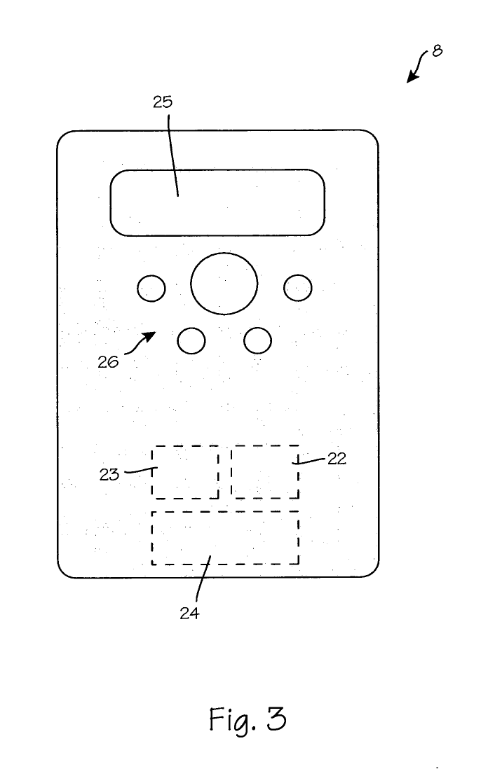

The data acquisition and recording module 8 is illustrated in FIG. 3. The module comprises a wireless receiver 22, a control system 23 having a microprocessor, and a data storage device 24 capable of storing digital data. The module may further comprise a display 25 and user interface 26. The user interface may be used to program the device and provide information to the control system 23. The module is capable of receiving and recording data sent from the indwelling sensor array. The control system may be provided with time and event markers to indicate and record the occurrence of an event when the wireless ambulatory reflux monitoring system is in use. An event is the patient's perception that reflux is occurring. The user interface is accessible by the patient and is operable to accept user input to mark events at the discretion of the user. Thus a user can mark any perceived instance of reflux. The module is sized and dimensioned to be portable and easily worn by a patient. The module may further be provided with a modem and wireless transmitter or an Ethernet© or USB© port so it may be placed in electrical communication with a computer where the data can be further analyzed by software.

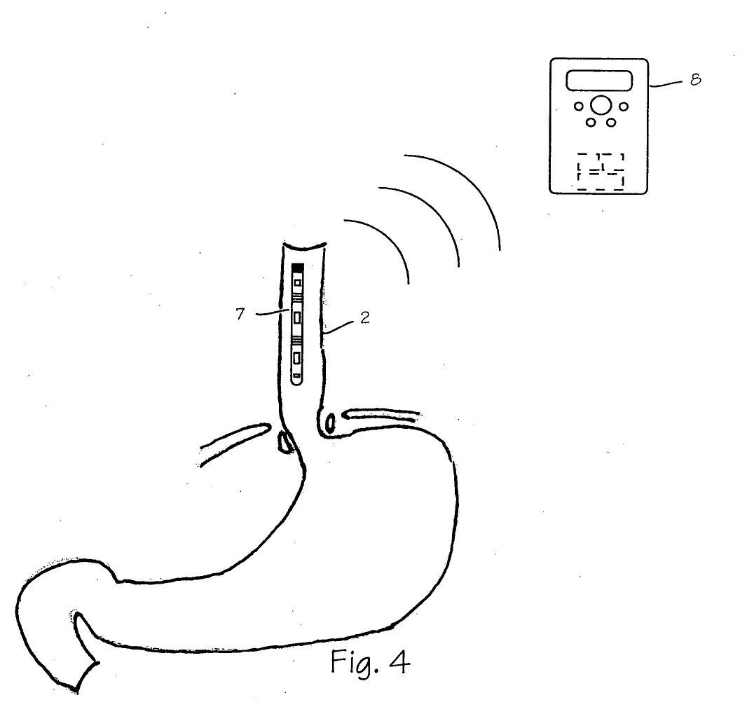

FIG. 4 illustrates the wireless ambulatory reflux monitoring system in use. The indwelling sensor array is inserted through the patient's mouth by the doctor and lowered down the patient's esophagus. Readings from the pressure sensors are taken to determine the proper placement of the indwelling sensor array. Once the doctor determines the indwelling sensor array is located properly, the indwelling sensor array is attached to the wall of the esophagus. The array 7 is activated and the module 8 is held in proximity while the patient partakes in normal daily activity. The system is operated for an extended period as necessary to obtain data pertaining to esophageal function over an ambulatory time period. (By “ambulatory time period” we mean a suitable time period that is sufficiently likely to record events of diagnostic value while the patient is free to move about in normal daily activity. Preferably, the time period is about 24 hours to 72 hours, so that circadian conditions may be observed, and periods of several days may be useful to detect recurrent circadian patterns in esophageal function.) However, the array may be attached to the patient for longer or shorter periods of time depending on the needs of the patient and recommendations by the doctor. The patient is instructed to operate the interface, during the monitoring and recording of the biological parameters of the esophagus, to mark perceived instances of reflux, chest pains, spasms, etc. Further, the array sends data to the module during the ambulatory period independent of the Data from indwelling sensor array is sent wirelessly from the indwelling sensor array to the to the data acquisition and recording module. Unlike traditional esophageal pressure testing methods that last only 20 to 40 minutes, the wireless ambulatory reflux monitoring system allows for continuous 24 hour monitoring of pressure and other biological parameters within the esophagus. After the ambulatory period, the sensor array 7 is removed and the data recorded in the acquisition and recording module is analyzed to diagnose reflux symptoms and esophagus function.

Doctors may evaluate the data (both the biological parameters and the patient's input regarding perceived events) to determine the cause of GERD, to evaluate severity of GERD, to determine appropriate treatment, and to seek patterns of biological parameters that correlate to specific mechanisms of GERD. By reviewing data collected over an extended time frame, after the patient has escaped the stress of a limited supervised esophageal manometry test, will enable collection of pertinent data for a large class of patients that cannot recreate GERD symptoms while being observed or otherwise attended by their doctor. Further, by reviewing biological parameter data collected during an ambulatory time period and correlating this with the patient's marked events, the absence of GERD can be confirmed for some patients that perceive GERD (the perceived symptoms may be further explored to determine the true cause of such perceived incontinence). By reviewing pressure, pH, and impedance data correlated over ambulatory periods, differential diagnoses may be obtained, such as distinguishing failure of the internal sphincter from mechanical stresses on the esophagus, or improper functioning of the nerves controlling the sphincter. These differential diagnoses may not be possible in the typical short term pressure measurements.

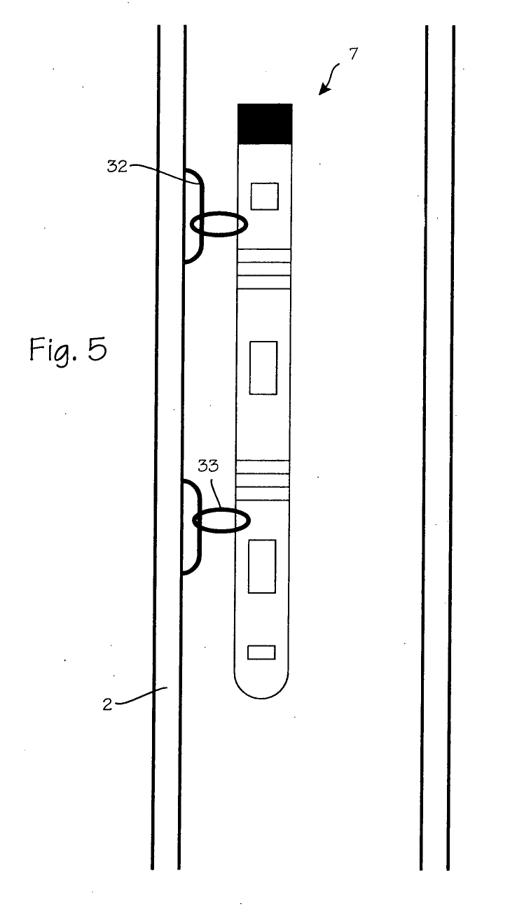

The indwelling sensor array may be attached to the esophagus through a variety of methods. As illustrated in FIG. 5, fixation devices 32 are inserted into the lining of the esophagus and rings or stitches 33 couple the indwelling sensor array to the fixation devices and the esophagus wall. Alternatively, the indwelling sensor array may be attached using other means such as stitches, barbs, staples or T-rings.

Unlike traditional supine esophageal pressure sensing systems and pH sensing systems that must be used serially in different time periods and under dissimilar testing conditions, the wireless ambulatory reflux monitoring system allows for the sensing of esophageal pressure, impedance and pH concurrently in the same time period and under the same conditions. This allows for data to be sensed and recorded in a normal day's setting. The wireless ambulatory reflux monitoring system can sense and record data during daily activities such as working, smoking, eating, drinking, sleeping and exercising. Additionally, observation of pH, pressure and impedance over the extended period will assist in the diagnosis of conditions evident only from the inspection of concurrent analysis of the data, and diagnosis of circadian patterns of esophageal function. These conditions may include reflux occurring only with the intake of specific foods by a patient. Monitoring and recording esophageal pressure, impedance and pH concurrently over extended periods will allow reflux to be properly diagnosed and distinguished from other physiological episodes occurring in a patient such as coughing, chronic chest pains, muscle spasms, regurgitation or neurological disorders.

Because the indwelling sensor array is in the esophagus for an extended period, the doctor will be able to detect abnormalities such as peristlasis, hammerlock or dysphagia in a natural (non-office/hospital) environment. As a result, more accurate diagnosis and therapies may be prescribed, as some of these abnormalities may not occur while a patient is in a doctor's office or hospital. Further, use of wireless ambulatory reflux monitoring system provides information about the strength and coordination of esophageal contractions and motor activity and the length, location, resting tone, and relaxing ability of the lower esophageal sphincter (LES). This information can be used to assess patients prior to anti-reflux surgery and disqualify those that are more likely to have a poor surgical outcome due to inadequate distal esophageal contractile strength, post-operative dysphagia, or inadequate LES relaxation. This information can also be used to determine if symptoms such as coughing, chest pains, spasms or regurgitation are causing reflux or if reflux is causing these symptoms.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A wireless ambulatory reflux monitoring system comprising:
an indwelling sensor array comprising a flexible shaft characterized by a distal section and proximal section and a control system disposed within the shaft, said distal section having a first electrode pair in electrical communication with the control system, a first pressure sensor in electrical communication with the control system and a pH sensor in electrical communication with the control system and said proximal section having a second electrode pair in electrical communication with the control system, a second pressure sensor in electrical communication with the control system and a wireless transmitter in electrical communication with the control system; and
a data acquisition and recording module in wireless communication with the indwelling sensor array, said module comprising a wireless receiver for receiving biological data sent by the indwelling sensor array and a memory device for recording data received by the wireless receiver.

2. The wireless ambulatory reflux monitoring system of claim 1 wherein the first or second pressure sensor comprise a solid state or microchannel transducer.

3. The wireless ambulatory reflux monitoring system of claim 1 further comprising a means for recording reflux events at the discretion of the patient contemporaneously with the recording of biological data

4. A method for recording esophageal data comprising:
providing an indwelling sensor array adapted for placement within an esophagus of a patient, said array further adapted to take pressure measurements, pH measurements, and impedance measurements within the esophagus and to transmit measurement data wirelessly;
placing the indwelling sensor array within the esophagus of the patient;
taking measurement data including pressure measurements, pH measurements, and impedance measurements within the esophagus using the indwelling sensor array;
sending measurement data wirelessly from the indwelling sensor array to a data acquisition and recording module; and
recording the measurement data.

5. The method of claim 4, wherein the indwelling sensor array comprises a flexible shaft having a first pressure sensor, a first electrode pair, and a pH sensor.

6. The method of claim 4, wherein the data acquisition and recording module comprises a wireless receiver for receiving data sent by the indwelling sensor array and a memory device for recording data received by the wireless receiver.

7. The method of claim 4 further comprising the step of indicating the occurrence of a reflux event.

8. The method of claim 4 wherein the step of recording the measurement data occurs over an ambulatory period.

9. The method of claim 4 further comprising the step of analyzing the measurement data to distinguish between reflux and other symptoms.

10. The method of claim 4 wherein the data acquisition and recording module further comprises means for recording reflux events at the discretion of the patient contemporaneously with the recording of measurement data.
